(19) United States
(12) Patent Application Publication
(10) Pub. No.: US 2023/0226547 A1
(43) Pub. Date: Jul. 20, 2023

(54) MICROFLUIDIC VALVE

(71) Applicant: Kilobaser GmbH, Graz (AT)

(72) Inventors: Alexander MURER, Graz (AT); Bernhard TITTELBACH, Graz (AT); Martin JOST, Graz (AT); Reinhard DIETHARDT, Weißkirchen (AT)

(73) Assignee: Kilobaser GmbH, Graz (AT)

(21) Appl. No.: 17/923,280

(22) PCT Filed: May 5, 2021
(86) PCT No.: PCT/EP2021/061813

§ 371 Date: Nov. 4, 2022

(30) Foreign Application Priority Data
May 6, 2020 (EP) 20173100.7

(51) Int. Cl. B01L 3/00; C07H 1/00; F16K 99/00; C07H 21/04

(52) U.S. Cl. CPC B01L 3/502738; C07H 1/00; F16K 99/0015; C07H 21/04; F16K 99/0055; B01L 2300/0864; B01L 2400/0655; B01L 2300/0816

(57) ABSTRACT

A microfluidic valve includes a carrier layer and a flexible membrane layer arranged on a surface of the carrier layer. The surface of the carrier layer has a valve chamber in the form of a spherical cap and a membrane formed by the flexible membrane layer covers at least the valve chamber. A plurality of microfluidic channels opening into the valve chamber are formed in the surface of the carrier layer. Moreover, an inflow channel and an outflow channel are connected to one another by a microfluidic connection channel. The connection channel and the valve chamber are positioned relative to each other in such a way that in the closed state of the membrane, a fluid can flow from the inflow channel via the connection channel into the outflow channel to bridge the valve chamber, while the at least one supply channel is closed by the membrane.

### FIELD OF INVENTION

The invention relates to a microfluidic valve comprising a carrier layer and a flexible membrane layer arranged on a surface of the carrier layer,

wherein the surface of the carrier layer has a valve chamber in the form of a spherical cap and a membrane formed by the flexible membrane layer covers at least the valve chamber, wherein a plurality of microfluidic channels opening into the valve chamber are formed in the surface of the carrier layer.

### DESCRIPTION OF THE PRIOR ART

In microfluidic systems, especially in microfluidic chips, the inherently known microfluidic behavior of liquids is exploited, wherein the clear diameter of the microfluidic channels of the system is usually less than one millimeter (1 mm) and usually between 100 nanometers (100 nm) and 500 micrometers (500 μm). In this case, flows with particularly low Reynolds numbers are formed, which corresponds to an almost exclusively laminar flow. Such microfluidic systems are used in particular for miniaturization, automation and improved integration of macrofluidic systems known per se.

Microfluidic valves are used in such microfluidic systems to regulate flow, in particular to shut off or release flow through a microfluidic channel and/or to seal off individual areas of the system at least temporarily from other areas.

Actuation of microfluidic valves can be based on a variety of different engineering principles, such as piezoelectric elements, magnetic components, electrical components, or pressurization or pressure change.

A favorable design of a microfluidic valve is characterized by a substantially rigid carrier layer, in the surface of which the microfluidic channels, namely an inflow channel and an outflow channel, are formed, wherein a flexible membrane layer is applied to the surface of the carrier layer. Furthermore, a valve chamber in the form of a spherical cap is provided in the surface of the carrier layer, wherein the spherical cap corresponds at most to a hemisphere. Both the inflow channel and the outflow channel open into the valve chamber. In the area of the valve chamber, the membrane layer covers the valve chamber as a membrane.

If the membrane is pressurized and thus brought into a closed state, the valve chamber is closed so that no fluid can pass from the inflow channel via the valve chamber into the outflow channel. If the pressure on the membrane is reduced and the membrane is thus brought into an open state, the membrane relaxes and assumes its original shape so that the valve chamber is unblocked and fluid can flow from the inflow channel via the valve chamber into the outflow channel.

While the simple design of such a microfluidic valve allows economical production, such microfluidic valves are difficult or costly to integrate into more complex microfluidic systems in which different microfluidic paths are to be alternately opened and closed by the corresponding valve position. Due to the fact that the microfluidic valve has only one open and one closed state, corresponding branch channels are necessary to connect the valve to the microfluidic system. In particular, this problem arises in microfluidic synthesis chips where a variety of different reagents need to be fed to a synthesis chamber in a predefined order, with one microfluidic valve controlling the feed of one reagent at a time.

### OBJECT OF THE INVENTION

It is therefore an object of the invention to overcome the disadvantages of the prior art and to propose a microfluidic valve that combines the advantages of economical manufacturing with ease of integration into a microfluidic system, in particular into a microfluidic chip.

### SUMMARY OF THE INVENTION

This object is solved in a microfluidic valve of the type mentioned above according to the invention in that the microfluidic channels comprise an inflow channel, an outflow channel and at least one supply channel,

wherein the microfluidic channels and the membrane are formed in such a way that the membrane can be brought into a closed state by application of pressure, in which closed state the membrane is pressed into the valve chamber to prevent the passage of a fluid to be introduced from the at least one supply channel into the valve chamber,

wherein the inflow channel and the outflow channel are connected to each other by a connection channel, wherein the connection channel and the valve chamber are positioned relative to each other in such a way that, in the closed state of the membrane, a fluid to be supplied can flow from the inflow channel via the connection channel into the outflow channel, while the at least one supply channel is closed by the membrane,

and that in an opened state of the membrane a fluid to be supplied can flow from the inflow channel and/or at least one fluid to be introduced can flow from the at least one supply channel into the valve chamber, wherein a fluid located in the valve chamber can flow out of the valve chamber via the outflow channel.

The design of the microfluidic valve according to the invention is characterized in that at least three microfluidic channels open into the valve chamber, namely both the inflow channel, the outflow channel and the at least one supply channel. When the membrane is in the open state and thus the valve chamber is unblocked, fluids can flow from the inflow channel and/or from the at least one supply channel into the outflow channel, depending on the applied pressure gradient. Although it is conceivable that fluids flow into the valve chamber from several channels simultaneously and flow out together via the outflow channel, it is preferred if the pressure gradients of the microfluidic channels are set in the operating state so that only one fluid path is flowed through.

Preferably, the microfluidic valve is designed in such a way that, when the membrane is open in the operating state, only one fluid to be introduced passes from the respective supply channel via the valve chamber into the outflow channel. However, it is also conceivable that fluids can flow simultaneously into the valve chamber from several supply channels.

Particularly preferably, exactly one supply channel is provided in order to achieve a particularly simple setup, wherein such a setup with three microfluidic channels is particularly suitable for feeding a reagent into a microfluidic system with several microfluidic valves connected in series.

Various plastics are particularly suitable as materials for the carrier layer, preferably selected so that they do not react with the fluids passed through the microfluidic valve, wherein the carrier layer preferably has a higher stiffness than the flexible membrane layer. The higher stiffness can be achieved, for example, by a correspondingly higher layer thickness of the carrier layer compared to the membrane layer, especially if the carrier layer and the membrane layer are made of plastic. Of course, glass or glass-like materials are also conceivable as a material for the carrier layer.

Various plastics can also be used as materials for the flexible membrane layer, wherein flexibility can be achieved in particular by a low layer thickness and/or a high elastic deformability of the plastic. The flexibility has no effect, or only an extremely small effect, on the microfluidic channels which are closed by the flexible membrane layer due to the smaller area of contact compared to the valve chamber. The pressurization of the membrane can, for example, be mechanical or hydraulic, but preferably the membrane is pressurized pneumatically.

Due to the fact that the inflow channel is connected to the outflow channel by the connection channel and that the valve chamber and connection channel are designed in such a way that the membrane in the closed state blocks the at least one supply channel but not the connection channel ensures that in the closed state of the membrane or the microfluidic valve, fluid to be supplied can still flow through the microfluidic valve via the inflow channel, connection channel and outflow channel. In other words, the inflow channel and outflow channel are short-circuited by the connection channel and the valve chamber is bridged by the connection channel, respectively, so that the corresponding microfluidic valve fluid path between the inflow channel and outflow channel can still be flowed through even when the membrane is closed.

The design according to the invention allows the microfluidic valve to be integrated into a microfluidic system in a simple manner: Inflow channel, connection channel and outflow channel are used as a common flow channel, so that in the closed state of the membrane fluid can flow through the microfluidic valve without an additional microfluidic component, such as a branch line and a bypass line. Thus, the space requirement of a microfluidic valve according to the invention in a microfluidic system is also greatly reduced compared to a conventional microfluidic valve with additional microfluidic components.

In the open state of the membrane, the at least one supply channel is open and thus, depending on the number of supply channels, at least one additional fluid path is open. Accordingly, in the open state of the membrane, fluid to be introduced can pass from the supply channel or from one of the supply channels via the valve chamber into the outflow channel. In both valve states, the fluid fed through the microfluidic valve can be continued via the outflow channel. The great advantage of the microfluidic valve according to the invention becomes particularly clear when several microfluidic valves are connected to each other in series, wherein the outflow channel of one microfluidic valve is connected to the inflow channel of the subsequent microfluidic valve. For the sake of clarity, the following considerations refer to only one supply channel per microfluidic valve, although more than one supply channel per microfluidic valve can be provided, as explained above.

If all microfluidic valves are closed, fluid can flow unhindered from the first inflow channel to the last outflow channel via the unobstructed connection channels. However, if one of the microfluidic valves is open, fluid can flow from the supply channel of the open microfluidic valve through the connection channels of the downstream closed microfluidic valves to the last outflow channel.

It is advantageous for the through-flow and the integrability of the microfluidic valve if the inflow channel and outflow channel are arranged extending along a straight line, in particular parallel to the surface of the carrier layer. Likewise, it can be provided that the connection channel is also located on the straight line as seen from the membrane layer, i.e. as seen from above.

Accordingly, one or more microfluidic valves according to the invention can be integrated in a simple manner into microfluidic systems, in particular into microfluidic chips.

In one embodiment variant of the invention, the connection channel extends below the valve chamber in relation to the flexible membrane layer and is open in the direction of the valve chamber. The fact that the connection channel extends below the valve chamber makes it possible to ensure in a simple manner that the connection channel is not closed by the membrane when it is closed, but that the flow through it remains possible. By opening the connection channel in the direction of the valve chamber, a fluidic connection is established between the valve chamber and the connection channel, so that it is ensured that fluid can pass from the at least one supply channel into the connection channel when the membrane is in the open state, so that the fluid is also passed via the connection channel into the outflow channel. At the same time, it can thus be ensured in a simple manner that fluid present in the valve chamber can flow out of the valve chamber via the connection channel during closure of the membrane.

In order to enable particularly simple production of the microfluidic valve, with all microfluidic channels as well as the valve chamber and the microfluidic connection channel being produced with as few production steps as possible, preferably in a common production step, in particular by stamping or lithographic processes, it is provided according to a further embodiment variant of the invention that the connection channel is formed as a channel-shaped depression in the valve chamber. The connection channel thus extends from a boundary surface of the valve chamber into the carrier layer. Preferably, a depth of the channel-shaped depression is constant with respect to the boundary surface of the valve chamber. The cross-section of the channel-shaped depression can be selected as desired with the exception of the geometric shape predetermined by the boundary surface, wherein the channel-shaped depression can be designed, for example, in the form of a groove.

A further embodiment variant of the invention provides that the connection channel, preferably in the area of the valve chamber, is designed to extend in an arc between the inflow channel and the outflow channel with respect to the flexible membrane layer. While the inflow channel, connection channel and outflow channel preferably extend in a straight line when viewed from the direction of the membrane layer—i.e. from above—in order to reduce flow resistance, the connection channel in this embodiment variant extends in an arc between the inflow channel and outflow channel when viewed in cross section—i.e. from the side. Advantageously, the connection channel is bent in such a way that the arc shape of the connection channel in the area of the valve chamber follows the circular arc specified by the spherical cap shape of the valve chamber.

In order to enable a constant flow of a fluid as far as possible without local changes in the flow rate of the fluid in the region of the connection channel when the membrane closes the valve chamber, according to a further embodiment variant of the invention it is provided that a flow cross-section of the inflow channel and of the outflow channel are of the same size and the connection channel is dimensioned in such a way that its flow cross-section in the closed state of the membrane is substantially constant and corresponds to the flow cross-section of the inflow channel and outflow channel.

Due to the outstanding integrability of the microfluidic valves according to the invention into a microfluidic system, the invention also relates to a microfluidic chip comprising a chip carrier layer and a flexible chip membrane layer applied to a surface of the chip carrier layer, wherein the chip carrier layer has a plurality of fluidic connections and a microfluidic channel system connected to the fluidic connections is formed in the surface of the chip carrier layer, wherein microfluidic valves are provided for flow regulation of the microfluidic channel system. In this regard, it is further provided that at least one of the microfluidic valves is formed as a microfluidic valve according to the invention, each having a supply channel, wherein the flexible membrane layer of the at least one microfluidic valve is formed by the flexible chip membrane layer,

wherein the carrier layer of the at least one microfluidic valve is formed by the chip carrier layer and the microfluidic channels of the at least one microfluidic valve are part of the microfluidic channel system,

wherein the supply channel of the at least one microfluidic valve is connected to one of the fluidic connections.

Since the carrier layer of the at least one microfluidic valve is formed by the chip carrier layer and the flexible membrane layer of the at least one microfluidic valve is formed by the flexible chip membrane layer, the at least one microfluidic valve is fully integrated into the microfluidic chip. In other words, the at least one microfluidic valve can be fabricated together with the microfluidic chip without requiring additional process steps or complex integration into the microfluidic channel system. The at least one microfluidic valve can be used as a metering valve to allow fluid to be introduced from the supply channel into the microfluidic channel system of the chip when the membrane is in the open state. In the closed state of the membrane, fluid can be fed through the microfluidic valve via the connection channel while the supply channel is closed.

Likewise, this design allows microfluidic valves with spherical cap-shaped valve chambers according to the prior art to be integrated into the microfluidic chip in a simple manner if simple blocking valves are required for the function.

As mentioned at the outset, the particular synergistic effect of the design of the microfluidic valves according to the invention is particularly apparent when more than one of the microfluidic valves is provided and the microfluidic valves are connected in series. Particularly advantageously, this can be used in a microfluidic chip configured to synthesize a compound, preferably an oligonucleotide, in a synthesis chamber, and preferably multiple reagents must be supplied to the synthesis chamber to perform the synthesis. For the purpose of transporting the reagents from the fluidic connections to the synthesis chamber, the microfluidic chip accordingly comprises a main channel. Thus, in one embodiment variant of the microfluidic chip, it is provided that the microfluidic chip comprises a synthesis chamber for synthesizing an oligonucleotide and a main conduit channel connected to the synthesis chamber, wherein a plurality of the microfluidic valves are formed as microfluidic valves, wherein the main conduit channel is formed at least in sections by the inflow channels, outflow channels and connection channels of the microfluidic valves. By integrating the microfluidic valves into the main channel itself, fluid can flow unimpeded through the main channel via the corresponding connection channels when the microfluidic valves are closed. The introduction of a fluid from a fluidic connection into the main channel via a microfluidic valve associated with that connection can be achieved in a simple manner by opening the corresponding microfluidic valve. The remaining microfluidic valves remain closed to allow flow.

In order to ensure that the introduction or metering of reagents in the microfluidic chip takes place exclusively via microfluidic valves and that a common main channel, which may, however, have several branches, is provided, a further embodiment variant provides that a plurality of the fluidic connections are designed as reagent connections for the supply of reagents from reagent containers connected to the respective fluidic connections to the synthesis chamber, wherein all reagent connections are connected to the main channel via a microfluidic valve in each case.

Particularly advantageous is the use of the microfluidic valve technology according to the invention in microfluidic chips intended for use in a device for the synthesis of oligonucleotides as described in WO 2018/153999. Here, the synthesis of an oligonucleotide, preferably a DNA strand, with a predefinable sequence order and length is carried out by means of phosphoramidite synthesis, for which a plurality of reagents is required. Accordingly, a particularly preferred embodiment variant provides that the microfluidic chip is designed in such a way that it can be used in an automated device for the synthesis of oligonucleotides, preferably of DNA strands, with a predefinable length and sequence order by means of phosphoramidite synthesis, wherein the valve position of the microfluidic valves, in particular of the microfluidic valves, of the microfluidic chip can be controlled by means of the device.

### BRIEF DESCRIPTION OF THE FIGURES

The invention will now be explained in more detail by means of an exemplary embodiment. The drawings are exemplary and are intended to illustrate the idea of the invention, but in no way to restrict it or even to reproduce it conclusively.

The drawings show as follows:

FIG. 1 shows an isometric view of a microfluidic valve with a supply channel;

FIG. 2a shows a schematic sectional view of the microfluidic valve in an open state;

FIG. 2b shows a schematic sectional view of the microfluidic valve in an open state;

FIG. 3 shows a chip layout of a microfluidic chip in which a plurality of microfluidic valves are integrated.

### DETAILED DESCRIPTION OF THE PREFERRED EMBODIMENTS

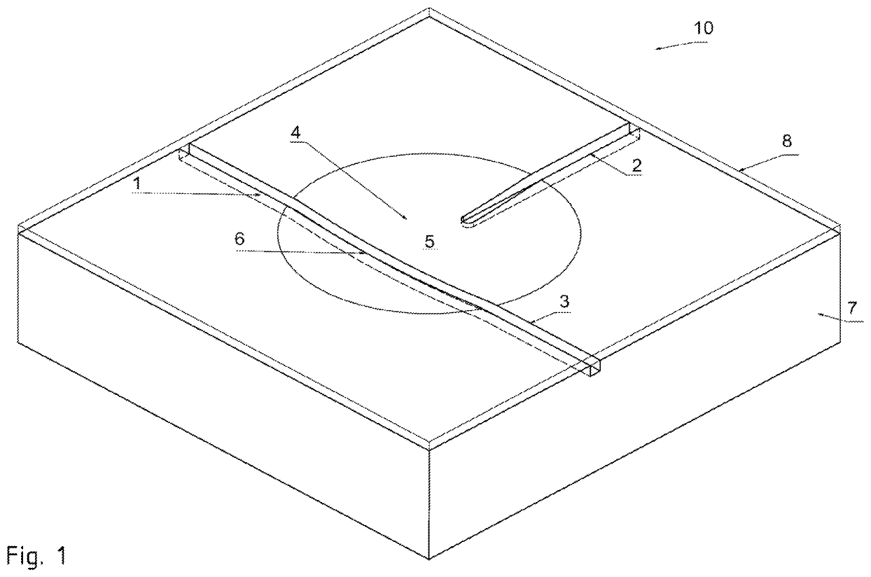

FIG. 1 shows an enlarged schematic representation of an exemplary embodiment of a microfluidic valve 10 according to the invention in an isometric view. The microfluidic valve 10 consists of a carrier layer 7 and a flexible membrane layer 8 applied directly to the carrier layer 7, which membrane layer 8 completely covers an upper side of the carrier layer 7, in the present exemplary embodiment.

A plurality of microfluidic channels 1,2,3,6 and a valve chamber 5 in the form of a spherical cap are formed in a surface of the carrier layer 7 forming the upper side. These structures can, for example, be embossed into the carrier layer 7 or produced by means of a lithographic process.

In the area of the valve chamber 5, the flexible membrane layer 8 forms a flexible membrane 4, which can be pressed into the valve chamber 5 by applying pressure.

The microfluidic channels 1,2,3,6 comprise an inflow channel 1 and an outflow channel 3, as well as a supply channel 2, all of which open into the valve chamber 5.

If the microfluidic valve 10 is viewed from above, i.e. along a vertical axis, so that the outline of the valve chamber 5 appears circular, then inflow channel 1 and outflow channel 3 are arranged in the present exemplary embodiment extending along a straight line, wherein the straight line extends as a secant through the circular outline of the valve chamber 5. In this view, the supply channel 2 extends radially in the direction of the imaginary center of the circular outline of the valve chamber 5 and transversely to the straight line defined by the inflow channel 1 and outflow channel 3. In the present exemplary embodiment, the straight line defined by inflow channel 1 and outflow channel 3 encloses a right angle with supply channel 2, wherein supply channel 2 is not arranged in the circular segment separated by the secant.

In addition, a microfluidic connection channel 6 is provided, which connects the inflow channel 1 and the outflow channel 3 with each other, wherein the connection channel 6 is formed as a channel-shaped depression in the valve chamber 5, which extends through the valve chamber 5 as a secant when viewed from above. In the present exemplary embodiment, the connection channel lies on the same straight line as inflow channel 1 and outflow channel 3.

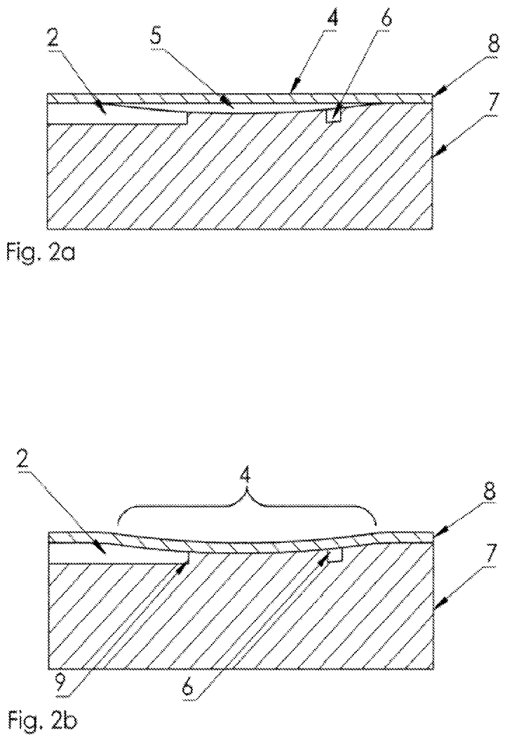

The operation of the microfluidic valve 10 is clear from FIGS. 2a and 2b. In FIG. 2a, the membrane 4 is relaxed and the valve chamber 5 is thus released. Thus, basically three fluid paths are possible: firstly, fluid from the inflow channel 1 can enter the outflow channel 3 via the released valve chamber 5, secondly, fluid from the supply channel 2 can enter the outflow channel 3 via the valve chamber 5, and thirdly, fluids from inflow channel 1 and supply channel 2 can also enter the valve chamber 5 simultaneously and flow off together via the outflow channel 3.

The supply channel 2 extends into the valve chamber 5 in the radial direction, wherein a channel depth (measured in the vertical direction from the relaxed membrane layer 8) of the supply channel 2 is greater than a depth of the valve chamber 5 at each point where the supply channel 2 ends in the valve chamber 5 in the radial direction. Thus, a sealing edge 9 is formed and it is achieved that fluid present in the valve chamber 5, which originates from the supply channel 2, can be forced back into the supply channel 2 when the membrane 4 is closed or can easily flow into the valve chamber 5 when it is opened.

As can also be seen, the connection channel 6 extends below the valve chamber 5 with respect to the membrane layer 8 and is open in the direction of the valve chamber 5.

This design of valve chamber 5, supply channel 2 and connection channel 6 ensures that the membrane 4—as shown in FIG. 2b—can be brought into a closed state by applying pressure, in which closed state the membrane 4 is pressed into the valve chamber 5 in order to prevent the transfer of a fluid to be introduced from the supply channel 2 into the valve chamber 5, while a fluid to be supplied can flow from the inflow channel 1 via the connection channel 6 into the outflow channel 3, although the valve chamber 5 is closed or filled by the membrane 4. The membrane 4 also forms an upper boundary of the connection channel 6 that is open in the direction of the valve chamber 5.

In other words, with the microfluidic valve 10 discussed, it is possible that when the membrane 4 is closed, the valve chamber 5 is bridged by the connection channel 6 to allow fluid transport from the inflow channel 1 to the outflow channel 3. Accordingly, when the membrane 4 is closed, the fluid path between the inflow channel 1 and the outflow channel 3 is open via the connection channel 6.

As can also be seen, the membrane 4 is pressed against the boundary surface of the valve chamber 5 in the closed state and thus, on the one hand, closes the supply channel 2, preferably via the circumferential sealing edge 9, and, on the other hand, prevents the direct transfer of fluid from inflow channel 1 or outflow channel 3 into the (closed) valve chamber 5.

In a section (not shown) through inflow channel 1, connection channel 6 and outflow channel 3 parallel to the vertical direction, connection channel 6 extends in an arc between inflow channel 1 and outflow channel 3.

The dimensions or the geometric design of the connection channel 6 is preferably selected so that a flow cross-section of the inflow channel 1, connection channel 6 and outflow channel 3 is essentially constant in the closed state of the membrane 4, i.e. when the membrane 4 closes the connection channel 6 upwards.

Although only one supply channel 2 is provided in the previously described exemplary embodiment, it is also conceivable to provide two or more supply channels 2 opening into the valve chamber 5 to create additional fluid paths.

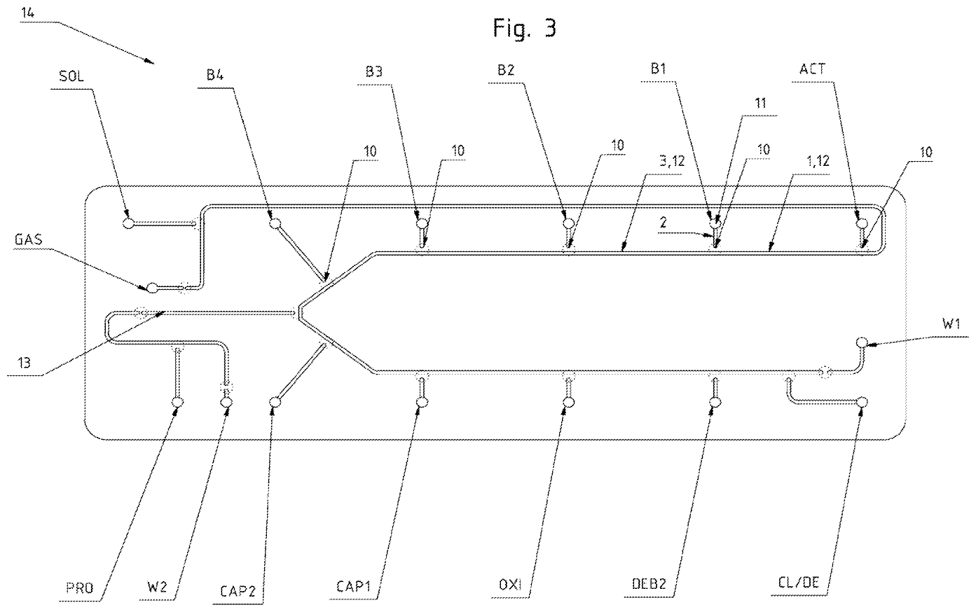

FIG. 3 shows a possible layout of a microfluidic chip 14 that allows synthesis of oligonucleotides in an automated device, wherein several of the microfluidic valves of the microfluidic chip 14 are designed as microfluidic valves 10.

The microfluidic chip 14 has a plurality of fluidic connections 11, wherein those fluidic connections 11 are connected to a main conduit channel 12 of the microfluidic chip 14 via a microfluidic valve 10 for supplying a reagent necessary for synthesis. Additionally, conventional microfluidic valves may also be provided, with the main conduit channel 12 being connected to a synthesis chamber 13. The synthesis chamber 14 contains a carrier medium with linker molecules, which act as a starting point for the synthesis of the oligonucleotides. The microfluidic valves 10, and preferably the other microfluidic valves, are fully integrated into the microfluidic chip 14 and are formed integrally. This is achieved by forming the valve chambers 5 and the microfluidic channels 1,2,3,6 of the microfluidic valves 10 in a chip carrier layer and thus the carrier layer 7 is formed by the chip carrier layer. Also, the flexible membrane layer 8 of the microfluidic valves 10 is formed by a chip membrane layer.

How the flow through the fluidic connection 13 of the microfluidic chip 10 is accomplished is described in the following on the basis of the first base B1. This principle can be applied analogously to all other reagents.

The fluidic connection 11 of the first base B1 is connected to a supply channel 2 of the corresponding microfluidic valve 10. The inflow channel 1, the connection channel 6 and the outflow channel 3 are formed as part of the main conduit channel 12 of the chip 14. When the microfluidic valve 10 associated with the first base B1 is closed, fluid from fluidic connections 11 further away from the synthesis chamber 13, for example solvent SOL or reagent for activating a detritylated 5′-OH group ACT, can flow through the microfluidic valve 10 associated with the first base B1 or through the section of the main conduit channel 12 formed by this microfluidic valve 10 when the microfluidic valve 10 is correspondingly open, without this fluid mixing with the first base B1.

If the microfluidic valve 10 associated with the first base B1 is open, the first base B1 can enter the main conduit channel 12 via the supply channel 2 and the valve chamber 5 of the microfluidic valve 10 and subsequently flows through the downstream, closed microfluidic valves 10.

In order to enable the flow and to establish corresponding pressure conditions, a microfluidic valve is additionally opened, which controls a fluidic outlet connection formed as a second outlet W2. The second outlet W2 is arranged downstream of the synthesis chamber 13 in the direction of flow, so that the first base B1 flows to the second outlet W2 via the main conduit channel 12 and the synthesis chamber 13. The second outlet W2 can be connected to a waste container, for example.

The microfluidic valves and the microfluidic valves 10 are basically kept in a closed position, so that the supply channels 2 associated with the reagents are blocked and only the main conduit channel 12 is open. The closed position is achieved, for example, by a control device of an automated and programmable synthesis device applying pressure to the membranes 4 of the microfluidic valves 10 or to the microfluidic valves via a pneumatic system, such as via control lines 28. If the pressure exerted on one of the microfluidic valves or microfluidic valves 10 is reduced by the control device or if the pressurization is suspended, the corresponding microfluidic valve or microfluidic valve 10 opens and the fluidic connection between the corresponding supply channel 2 and the main conduit channel 12 is established or open.

Before the synthesis can start, it is necessary to supply the reagents from reagent containers to the microfluidic valves 10 via transport lines. Therefore, the microfluidic valves 10 for the fluidic connections 11 of the reagents required for the synthesis are opened in sequence, one at a time, together with the microfluidic valve for a first outlet W1. Between the individual feeds, the main conduit channel 12 is first flushed in each case by simultaneously opening the microfluidic valve 10 for the solvent SOL and the microfluidic valve for the first outlet W1, and then dried by opening the microfluidic valve for the inert gas GAS and the microfluidic valve for the first outlet W1. As a result, none of the reagents enters the synthesis chamber 13 during the feeding, rinsing and drying processes. By simultaneously opening the microfluidic valve for the inert gas GAS and one of the microfluidic valves 10 for each of the fluidic connections 11 of the reagents required for the synthesis, the reagents can be conveyed back to the reagent containers after the synthesis has ended.

In the following, the synthesis steps of a synthesis cycle will be discussed, which are necessary for coupling a nucleotide to the end of a partial sequence of an oligonucleotide or as the first nucleotide to a linker molecule of a carrier medium. The sequence of the synthesis steps and the reagents used for them are known per se.

Each oligonucleotide starts at a linker molecule of a carrier medium located in the synthesis chamber 13 and is extended with each synthesis step by one nucleotide, which is coupled to the end of the chain. The 5′-OH group of the oligonucleotide is provided with an acid-labile dimethoxytrityl protecting group (4,4′-dimethoxytrityl—DMT).

First, a reagent for detritylation DEBL of one end of an oligonucleotide containing a partial sequence or for detritylation of the linker molecule is supplied to the synthesis chamber 13 from the corresponding reagent container. In this process, the microfluidic valve 10 controlling the fluidic connection 11 for the reagent for detritylation DEBL is opened together with the microfluidic valve controlling the second outlet W2, as described above.

This removes the DMT protecting group so that another nucleotide can be coupled to the free 5′-OH group. In the present case, the reagent for detritylation DEBL is an acidic solution, namely a solution containing 2% trichloroacetic acid or 3% dichloroacetic acid in an inert solvent such as dichloromethane or toluene. This step is also referred to as the deblocking step.

In the next step, the nucleotide chain is extended at the detritylated free 5′-OH group by one base, i.e. either adenine B1, guanine B2, cytosine B3 or thymine B4 for a DNA strand or uracil B4 for an RNA strand. For this purpose, a reagent for activating ACT of the free 5′-OH group and a reagent containing phosphoramidite of the corresponding base B1,B2,B3,B4 are alternately supplied to the synthesis chamber 14. The phosporamidites are fed dissolved in a solvent SOL, in particular in acetonitrile. In the present case, activation of the 5′-OH group is achieved by means of a 0.2-0.7 molar solution of an acidic azole catalyst, in particular by 1H-tetrazole, 5-ethylthio-1H-tetrazole, 2-benzylthiotetrazole or 4,5-dicyanoimidazole. In this process, the nucleotide couples to the free 5′-OH group of the oligonucleotide, while the phosphoramidite residue is cleaved off. The 5′-OH group of the newly coupled nucleotide is again protected by a DMT protecting group. This step is also referred to as the coupling step.

In the synthesis chamber 13, a mixture of two reagents for blocking CAP1,CAP2 unreacted 5′-OH groups is added in the next step. In the present case, blocking of unreacted 5′-OH groups is achieved by a mixture of acetic anhydride and 1-methylimidazole as catalyst. This step is also referred to as the capping step.

The final step in a synthesis cycle is the oxidation of a phosphite triester bond formed between the newly coupled nucleotide and the corresponding 5′-OH group of the oligonucleotide by adding a reagent for oxidation OXI. The reagent for oxidation OXI oxidizes the phosphite triester bond into a four-coordinated phosphotriester, a protected precursor of the naturally occurring phosphate diester internucleoside bond. This stabilizes the bond between the coupled nucleotide and the corresponding 5′-OH group. In the present case, oxidation is achieved under anhydrous conditions using (1S)-(+)-(10-camphersulfonyl)-oxaziridine (CSO). This step is referred to as the oxidation step.

The four steps of a synthesis cycle are repeated in the order of the nucleotide sequence of the oligonucleotide to be synthesized until the oligonucleotide has the predetermined length and sequence. The order of the nucleotide sequence can, for example, be predetermined by an appropriately automated device and can be set by a user, so that a plurality of different individually predeterminable oligonucleotides can be synthesized in a device with such a chip 14.

Once the oligonucleotide to be synthesized is completed, a reagent for cleaving the oligonucleotides from the linker molecules and for removing the protecting groups CL/DE is supplied to the synthesis chamber 13. In the present case, the reagent used for cleaving the oligonucleotides from the linker molecules and for removing the protecting groups CL/DE is a mixture of ammonia and methylamine, wherein the two reagents are preferably present in equal amounts in the mixture. This reagent CL/DE dissolves the oligonucleotides from the linker molecules, wherein this process takes about 3 to 15 minutes, usually about 5 minutes. During this process, the microfluidic valve controlling the fluidic connection 11 for a product collection vessel PRO is open to deliver the synthesized oligonucleotides into the product collection vessel PRO. Removal of the protecting groups typically takes an additional 3 to 15 minutes, typically about 5 minutes, when the product collection vessel PRO is brought to a temperature between 50° and 750, preferably about 65° C., for example via a built-in heating block. At room temperature, this process requires between 45 and 120 minutes, usually about 60 minutes.

After completion of the synthesis, the PRO product collection container can be removed and further processed.

It is understood that the described microfluidic valves 10 can be used in a variety of different microfluidic systems and microfluidic chips.

### LIST OF REFERENCE SIGNS

1 Inflow duct
2 Supply channel
3 Outflow channel
4 Membrane
5 Valve chamber
6 Connection channel
7 Carrier layer
8 Flexible membrane layer
9 Sealing edge
10 Microfluidic valve
11 Fluidic connection
12 Main conduit channel
13 Synthesis chamber
14 Microfluidic chip
SOL Solvent
GAS Inert gas
ACT Reagent for the activation of a detritylated 5′-OH group.
B1 Base 1 (e.g. phosphoramidite of the base adenine)
B2 Base 2 (e.g. phosphoramidite of the base guanine)
B3 Base 3 (e.g. phosphoramidite of the base cytosine)
B4 Base 4 (e.g. phosphoramidite of the base thymine or uracil)
W1 First outlet
CL/DE Reagent to cleave the oligonucleotides from the linker molecules and/or a reagent to remove the protecting groups
DEBL Reagent for the detritylation of a 5′-OH group provided with a dimethoxytrityl protecting group
OXI Reagent for the oxidation of a phosphite triester bond
CAP1 First reagent for blocking unreacted 5′-OH groups
CAP2 Second reagent for blocking unreacted 5′-OH groups
PRO Product collection container
W2 Second outlet

1. A microfluidic valve, comprising a carrier layer and a flexible membrane layer arranged on a surface of the carrier layer,
wherein the surface of the carrier layer has a valve chamber in the form of a spherical cap and a membrane formed by the flexible membrane layer covers at least the valve chamber,
wherein a plurality of microfluidic channels opening into the valve chamber are formed in the surface of the carrier layer,
wherein the microfluidic channels comprise an inflow channel, an outflow channel and at least one supply channel,
wherein the microfluidic channels and the membrane are formed in such a manner that the membrane can be brought into a closed state by application of pressure, in which closed state the membrane is pressed into the valve chamber in order to prevent the transfer of a fluid to be introduced from the at least one supply channel into the valve chamber,
wherein the inlet channel and the outlet channel are connected to each other by a microfluidic connection channel,
wherein the connection channel and the valve chamber are positioned relative to each other in such a way that in a closed state of the membrane, a fluid to be supplied can flow from the inflow channel via the connection channel into the outflow channel, while the at least one supply channel is closed by the membrane,
wherein, in an open state of the membrane, a fluid to be supplied can flow from the inflow channel and/or at least one fluid to be introduced can flow from the at least one supply channel into the valve chamber, wherein a fluid located in the valve chamber is able to flow out of the valve chamber via the outflow channel,
wherein a flow cross-section of the inflow channel and of the outflow channel are of the same size and the connection channel is dimensioned in such a way that its flow cross-section is substantially constant in the closed state of the membrane and corresponds to the flow cross-section of the inflow channel and outflow channel.

2. The microfluidic valve according to claim 1, wherein the connection channel extends below the valve chamber with respect to the flexible membrane layer and is open in the direction of the valve chamber.

3. The microfluidic valve according to claim 1, wherein the connection channel is formed as a channel-shaped depression in the valve chamber.

4. The microfluidic valve according to claim 1, wherein the connection channel, preferably in the region of the valve chamber, is designed to extend in an arc shape between the inflow channel and the outflow channel with respect to the flexible membrane layer.

5. (canceled)

6. The microfluidic chip, comprising a chip carrier layer and a flexible chip membrane layer applied to a surface of the chip carrier layer,
wherein the chip carrier layer has a plurality of fluidic connectors and a microfluidic channel system connected to the fluidic connections is formed in the surface of the chip carrier layer,
wherein microfluidic valves are provided for flow regulation of the microfluidic channel system,
wherein at least one of the microfluidic valve elements is formed as a microfluidic valve according to claim 1 having one supply channel,
wherein the flexible membrane layer of the at least one microfluidic valve is formed by the flexible chip membrane layer,
wherein the carrier layer of the at least one microfluidic valve is formed by the chip carrier layer and the microfluidic channels of the at least one microfluidic valve are part of the microfluidic channel system,
wherein the supply channel of the at least one microfluidic valve is connected to one of the fluidic connectors.

7. The microfluidic chip according to claim 6, wherein the microfluidic chip has a synthesis chamber for synthesizing an oligonucleotide and a main conduit channel connected to the synthesis chamber,
wherein a plurality of the microfluidic valve elements is formed as microfluidic valves, wherein the main conduit channel is formed at least in sections by the inflow channels, outflow channels and connection channels of the microfluidic valves.

8. The microfluidic chip according to claim 7, wherein a plurality of the fluidic connectors is designed as reagent connectors for supplying reagents from reagent containers connected to the respective fluidic connectors to the synthesis chamber, wherein all reagent connectors are connected to the main channel via a respective microfluidic valve.

9. A method for using the microfluidic chip according to claim 6, in an automated synthesizing device for the synthesis of oligonucleotides of a predefinable length and sequence via of a phosphoramidite synthesis, wherein the valve position of the microfluidic valve elements of the microfluidic chip is controlled by the automated synthesizing device.

10. The microfluidic valve according to claim 4, wherein a section of the connection channel being located in the region of the valve chamber extends in an arc shape between the inflow channel and the outflow channel with respect to the flexible membrane layer.

11. The method according to claim 9, wherein the automated synthesizing device synthesizes DNA strands of a predefinable length and sequence via a phosphoramidite synthesis carried out on the microfluidic chip.

12. The method according to claim 9, wherein the valve position of the at least one microfluidic valve is controlled by the automated synthesizing device.
